(19) United States
(12) Patent Application Publication
(10) Pub. No.: US 2026/0165721 A1
(43) Pub. Date: Jun. 18, 2026

(54) DEVICES, SYSTEMS, AND METHODS FOR PERFORMING THROMBECTOMY PROCEDURES

(71) Applicant: THE BOARD OF TRUSTEES OF THE LELAND STANFORD JUNIOR UNIVERSITY, Stanford, CA (US)

(72) Inventors: Renee Zhao, Stanford, CA (US); Paul G. Yock, Atherton, CA (US); Yilong Chang, Stanford, CA (US); Jeremy J. Heit, Los Altos, CA (US)

(21) Appl. No.: 19/535,808

(22) Filed: Feb. 10, 2026

Related U.S. Application Data
(63) Continuation of application No. PCT/US2024/041855, filed on Aug. 11, 2024.
(60) Provisional application No. 63/532,279, filed on Aug. 11, 2023.

(51) Int. Cl. A61B 17/22; A61B 17/00; A61B 17/3207

(52) U.S. Cl. CPC A61B 17/22; A61B 17/00234; A61B 17/320758; A61B 2017/00305; A61B 2017/00398; A61B 2017/00544; A61B 2017/00561; A61B 2017/00778; A61B 2017/22079; A61B 2217/005

(57) ABSTRACT

Thrombectomy devices, systems, and methods are provided for performing thrombectomy procedures that include an aspiration catheter, a spinner device, and a source of vacuum, e.g., a pump for aspirating clot through the catheter. The spinner device includes a shaft including a proximal end coupled to a motor, a distal end carrying a spinner head positionable in a lumen of the catheter for debulking and/or otherwise removing clot adjacent an outlet in a distal end of the catheter. The catheter may be introduced into a body lumen to position the outlet adjacent a target clot, the source of vacuum may be activated, and, with the spinner head within the catheter adjacent the outlet, the motor may be activated to spin the spinner head to debulk and/or otherwise remove the clot through the catheter lumen around the spinner device.

### RELATED APPLICATION DATA

The present application is a continuation of co-pending International Application No. PCT/US2024/041855, filed Aug. 11, 2024, which claims benefit of U.S. provisional application Ser. No. 63/532,279, filed Aug. 11, 2023, the entire disclosures of which are expressly incorporated by reference herein.

### STATEMENT REGARDING FEDERALLY SPONSORED RESEARCH AND DEVELOPMENT

None.

### TECHNICAL FIELD

The present application relates to medical devices and, more particularly, to thrombectomy devices, and to systems and methods for performing thrombectomy or other medical procedures using such devices.

### BACKGROUND

Acute Ischemic Stroke (AIS) is the leading cause of disability and the fifth leading cause of death in the United States. AIS results from blockage or interruption of blood flow within a cervical or cerebral artery, and this lack of blood flow to the brain may result in irreversible brain injury (core infarction) or impaired neuronal function in ischemic, but potentially salvageable, brain tissue (penumbra). AIS may be treated with intravenous thrombolysis within 3-4.5 hours of symptom onset, but fewer than five percent of AIS patients reach medical care within this time frame.

More recently, endovascular thrombectomy using aspiration or/and a stent retriever device has been shown to be an effective treatment for AIS that involves large vessel occlusion (AIS-LVO) of the internal carotid, proximal middle cerebral, proximal anterior cerebral arteries, or basilar artery up to twenty four hours from symptom onset. Thrombectomy has led to a marked improvement in AIS-LVO patient outcomes, and it has become the standard of care for AIS patients with an AIS-LVO.

Despite the recent advancements in AIS treatment, there are significant gaps in knowledge and available devices that limit optimal treatment of AIS treatments. Current thrombectomy techniques fail to restore any or insufficient blood flow in about fifteen percent (15%) of patients after multiple passes, with aspiration methods having a failure rate of about twenty-five to thirty three percent (25-33%). Common reasons for failed thrombectomy include clots with high fibrin content, clot fragmentation that prevents complete removal, and clots that are resistant to modern thrombectomy devices. In addition, recent data indicate that in order to be maximally effective, thrombectomy should restore about ninety-five to one hundred percent (95-100%) of the blood flow distal to the site of arterial blockage, and this blood flow should be restored within one thrombectomy attempt (“first pass effect”) to maximize the likelihood of achieving a good outcome. However, the first pass effect is achieved in fewer than about fifty percent (50%) of patients who undergo thrombectomy, and there is a substantial need to develop new thrombectomy devices to improve the thrombectomy process.

Therefore, improved devices and methods for performing thrombectomy procedures would be useful.

### SUMMARY

The present application is directed to medical devices and, more particularly to thrombectomy devices, and to systems and methods for performing thrombectomy or other medical procedures using such devices.

In accordance with one example, a thrombectomy device is provided that includes an elongated tubular comprising a proximal end, a distal end sized for introduction into a body lumen of a patient, and a lumen extending between the proximal end and an outlet in the distal end; a source of vacuum coupled to the proximal end and communicating with the lumen; and a spinner device. The spinner device includes a shaft comprising a first end and a second end sized for introduction into the lumen, thereby defining a longitudinal axis therebetween; a spinner head on the second of the shaft positionable within the lumen adjacent the outlet within the lumen; and a motor coupled to the first end of the shaft for rotating the shaft and spinner head around the axis to debulk or otherwise remove clot adjacent the distal end.

In accordance with another example, a thrombectomy device is provided that includes an elongated tubular comprising a proximal end, a distal end sized for introduction into a body lumen of a patient, and a lumen extending between the proximal end and an outlet in the distal end; a source of vacuum coupled to the proximal end and communicating with the lumen; and a spinner device. The spinner device includes a shaft comprising a first end and a second end sized for introduction into the lumen, thereby defining a longitudinal axis therebetween; a spinner head on the second of the shaft positionable within the lumen and movable distally to extend the spinner head through the outlet into or through a target clot and proximally to pull the clot into the lumen; and a motor coupled to the first end of the shaft for rotating the shaft and spinner head around the axis to debulk or otherwise remove clot adjacent the distal end.

In accordance with still another example, a method is provided for performing thrombectomy that includes introducing a distal end of a tubular member into a body lumen adjacent a target blood clot; positioning a spinner head within a lumen of the tubular member adjacent an outlet in the distal end; activating a source of vacuum communicating with the lumen to aspirate material into the outlet; and rotating the spinner head to debulk and/or otherwise remove the clot into the outlet and aspirate the clot through the lumen.

In accordance with yet another example, a method for performing thrombectomy that includes introducing a distal end of a tubular member into a body lumen adjacent a target clot; activating a source of vacuum communicating with the lumen to aspirate material into the outlet; advancing a spinner head from within a lumen of the tubular member into or through the clot; retracting the spinner head back into the lumen to at least partially pull the clot into the lumen; and rotating the spinner head to debulk and/or otherwise remove the clot through the lumen.

Other aspects and features of the present invention will become apparent from consideration of the following description taken in conjunction with the accompanying drawings.

### BRIEF DESCRIPTION OF THE DRAWINGS

It is believed the present invention will be better understood from the following description of certain examples taken in conjunction with the accompanying drawings, in which like reference numerals identify the same elements and in which:

FIGS. 1A and 1B show an example of thrombectomy device including an aspiration catheter, a shaft carrying a spinner head, a controller for operating a motor to spin the shaft and spinner head, and a source of vacuum.

FIGS. 2A-2G show examples of spinner heads that may be included in the device of FIGS. 1A and 1B.

FIGS. 3A-3C show an exemplary method for performing a thrombectomy procedure using the device of FIGS. 1A and 1B.

FIG. 3D shows an example of a spinner head with clot captured in elements of the spinner head after a thrombectomy procedure.

FIGS. 4A-4D are images showing an exemplary method in which clot is debulked and removed from a body lumen using the device of FIGS. 1A and 1B.

The drawings are not intended to be limiting in any way, and it is contemplated that various examples of the invention may be carried out in a variety of other ways, including those not necessarily depicted in the drawings. The accompanying drawings incorporated in and forming a part of the specification illustrate several aspects of the present invention, and together with the description serve to explain the principles of the invention; it being understood, however, that this invention is not limited to the precise arrangements shown.

### DETAILED DESCRIPTION

The following description of certain examples of the invention should not be used to limit the scope of the present invention. Other examples, features, aspects, embodiments, and advantages of the invention will become apparent to those skilled in the art from the following description, which is by way of illustration, one of the best modes contemplated for carrying out the invention. As will be realized, the invention is capable of other different and obvious aspects, all without departing from the invention. Accordingly, the drawings and descriptions should be regarded as illustrative in nature and not restrictive.

Before the examples are described, it is to be understood that the invention is not limited to particular examples described, as such may, of course, vary. It is also to be understood that the terminology used herein is for the purpose of describing particular examples only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and exemplary methods and materials are now described.

It must be noted that as used herein and in the appended claims, the singular forms “a,” “an,” and “the” include plural referents unless the context clearly dictates otherwise. Thus, for example, reference to “a compound” includes a plurality of such compounds and reference to “the polymer” includes reference to one or more polymers and equivalents thereof known to those skilled in the art, and so forth.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

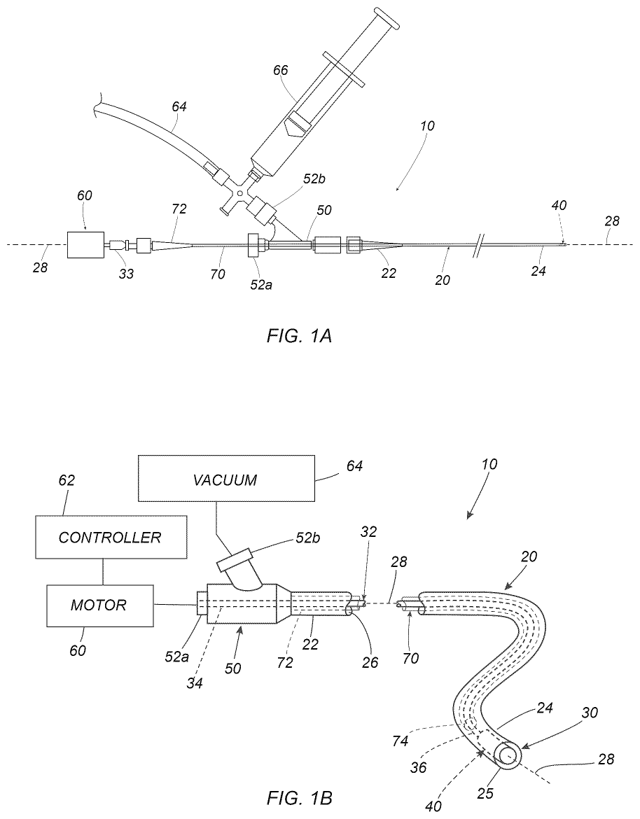

Turning to the drawings, FIGS. 1A and 1B show an example of a thrombectomy device or apparatus 10 that may be used with the systems and methods described herein. In general, the apparatus 10 includes an outer catheter, sheath, sleeve, or other tubular member 20, and a spinner device 30 including a spinner head or member 40 coupled to a shaft 32 that may be introduced through or otherwise positioned in the catheter 20. In addition, the apparatus 10 includes a source of vacuum 64, e.g., a pump, for aspirating material into and/or through a lumen 26 of the catheter 20. The apparatus 10 also includes a motor 60 coupled to the shaft 32 for rotating the shaft 32 and, consequently, the spinner head 40 and a controller 62 for operating the motor 60, as described elsewhere herein.

Optionally, the spinner device 30 may include an outer sleeve 70, which may be positioned and/or otherwise provided around the shaft 32. The sleeve 70 may help protect the inner surface of the catheter 20 when the shaft 32 rotates at high speeds, may reduce vibration resulting from rotation of the shaft 32 and/or spinner device 30, and/or may facilitate centering and/or stabilizing the spinner device 30 within the catheter 20.

Optionally, the catheter 20 and spinner device 30 may include one or more cooperating stops that limit distal movement of the spinner device 40 within the catheter 20, e.g., to prevent the spinner head 40 from being exposed beyond a distal end 24 of the catheter 20 and/or to ensure the spinner head 40 is positioned to achieve maximum clot debulking and/or removal, as described further elsewhere herein.

Generally, as shown, the catheter 20 is an elongate tubular member including a proximal end 22 including a handle or hub 50, a distal end 24 sized for introduction into a blood vessel or other body lumen, and one or more lumens 26 extending between the proximal and distal ends 22, 24, e.g., along a longitudinal axis 28. For example, as shown, a main lumen 26 may be provided that communicates with one or more ports 52 in the handle 50 and extends to an outlet 25 in the distal end 24. Optionally, the catheter 20 may include one or more additional lumens extending at least partially between the proximal and distal ends 22, 24, e.g., a guidewire lumen for receiving a guidewire or other rail, a steering element lumen, and the like (not shown).

The catheter 20 may be constructed using conventional biocompatible materials and/or methods, e.g., formed from plastic, various polymers, metal, composite materials, having a substantially homogenous construction between the proximal and distal ends 22, 24. Alternatively, the construction may vary along the length of the catheter 20 to provide desired properties, e.g., to provide a proximal portion that is substantially rigid or semi-rigid, e.g., providing sufficient column strength to allow the distal end 24 of the catheter 20 to be pushed or otherwise manipulated from the proximal end 22, while a distal portion adjacent the distal end 24 may be substantially flexible to facilitate advancement through tortuous anatomy. Optionally, the outer surface of the catheter 20 may also be coated or layered, e.g., with a lubricious material, if desired, to aid in advancement and/or other manipulation.

The distal end 24 of the catheter 20 may be sized to be introduced into a body lumen within a patient's body, e.g., into a cerebral or cervical artery or other location to perform a thrombectomy procedure. For example, the catheter 20 may have an outer diameter or other non-circular cross-section between about 1.5-2.5 millimeters, e.g., four to six French.

The shaft 32 of the spinner device 30 may be an elongated flexible member including a proximal end 34 and a distal end 36 sized to be received within the lumen 26 of the catheter 20. The shaft 32 may be sufficiently long to allow introduction of the distal end 36 into a target blood vessel, e.g., through or along with the catheter 20, while the proximal end 34 remains outside the patient's body. For example, the shaft 32 may be a solid or tubular cable, e.g., including a plurality of helically wound inelastic fibers, wires, and the like, constructed to translate rotation from the proximal end 34 to the distal end 36 to rotate the spinner head 40, e.g., with sufficient torsional strength such that rotation of the proximal end 34 causes directly corresponding rotation of the distal end 36, and consequently the spinner head 40 at relatively high speeds, when coupled to the motor 60, as described further elsewhere herein as described elsewhere herein.

The shaft 32 may have an outer diameter such that the shaft 32 may be positioned within the lumen 26 of the catheter 20 while providing sufficient clearance between the shaft 32 and the inner wall of the catheter 20 to allow clot or other debris to be aspirated through the lumen 26 around the shaft 32. For example, the shaft 32 may have an outer diameter between about 0.3 to one millimeter (0.3-1.0 mm) or about 0.5 millimeter.

Optionally, the sleeve 70 or other tubular member may be provided around the shaft 32, e.g., to prevent the shaft 32 from contacting the inner wall of the catheter 20 when introduced into the lumen 26 and/or rotated by the motor 60. For example, the sleeve 70 may include a lumen 76 extending between proximal and distal ends 72, 74 of the sleeve 70 and sized to receive the shaft 32 and allow the shaft 32 to rotate freely within the lumen without damage to the sleeve 70 or shaft 32. For example, if the shaft 32 has an outer diameter of 0.5 millimeter, the inner diameter of the lumen 76 may be 0.6 millimeter. The sleeve 70 may have an outer diameter smaller than the lumen 26 of the catheter 20, e.g., to ensure that aspiration vacuum within the catheter lumen 26 may be delivered to the distal end 24 of the catheter 20 and clot fragments or other debris may be removed through the catheter lumen 26 around the sleeve 70.

The sleeve 70 may have a substantially uniform construction between its proximal and distal ends. Alternatively, the construction may vary along the length of the sleeve 70. For example, a proximal portion of the sleeve 70 may be relatively stiff, e.g., to facilitate pushability of the sleeve 70, e.g., with or independent of the spinner device 30, and a distal portion of the sleeve 70 may relatively soft and/or flexible to enhance trackability through tortuous anatomy. In addition or alternatively, the distal end of the sleeve 70 immediately adjacent the spinner head 40 may be substantially stiff or otherwise reinforced to prevent axial compression or proximal displacement of the sleeve 70 when clot presses against the spinner head 40, e.g., to prevent proximal movement of the spinner head during operation of the device 10. Optionally, the sleeve 70 may be formed from lubricious material, e.g., PTFE, and/or may include a coating on an inner surface thereof to reduce friction and/or otherwise facilitate the shaft 32 rotating in the sleeve.

The sleeve 70 may be axially fixed relative to the shaft 32, e.g., such that the sleeve extends from the proximal end 34 to the distal end 36 immediately proximal to the spinner head 40. Alternatively, the sleeve 70 may be separate from the spinner device 30, e.g., such that the sleeve 70 may be introduced into the lumen 26 of the catheter 20 before introducing the spinner head 40 and shaft 32.

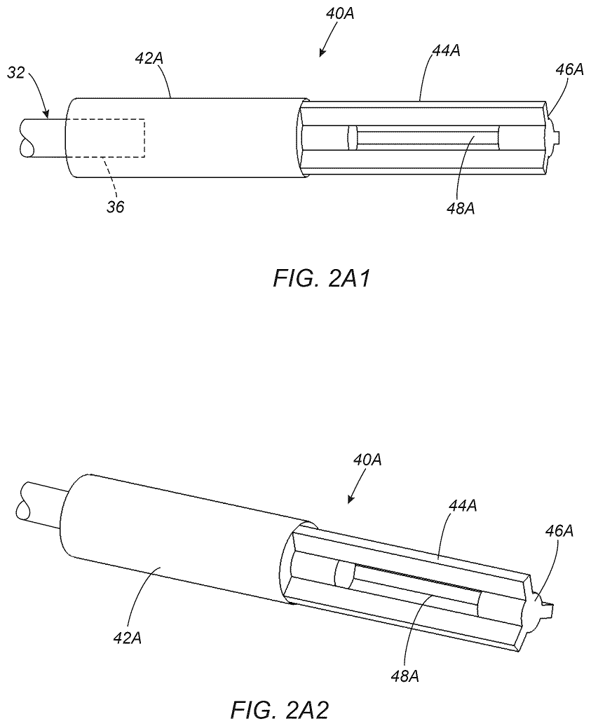

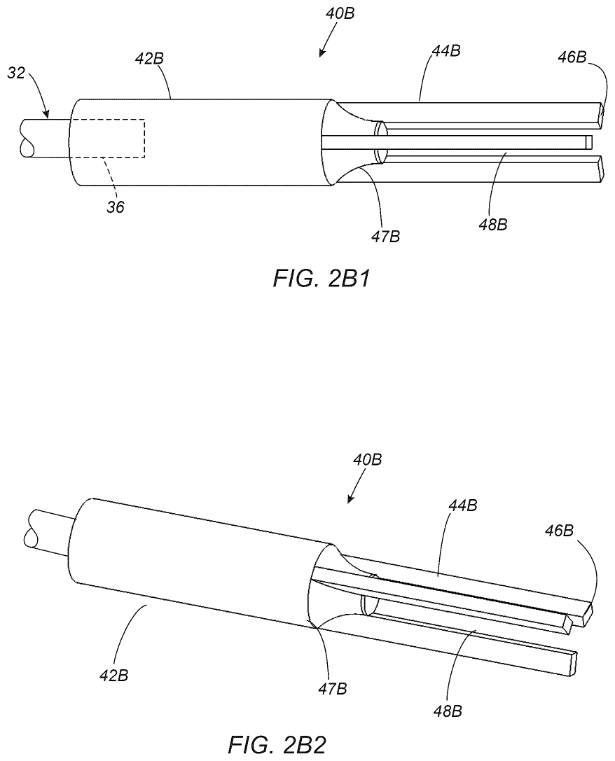

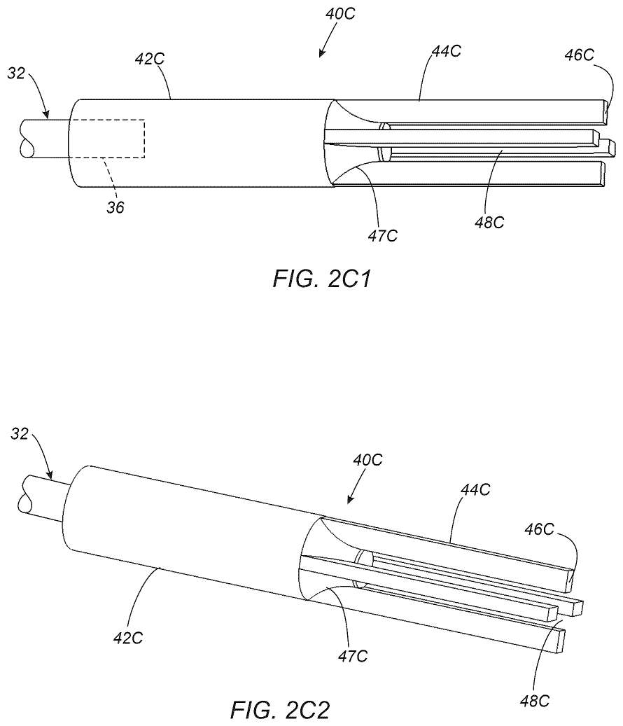

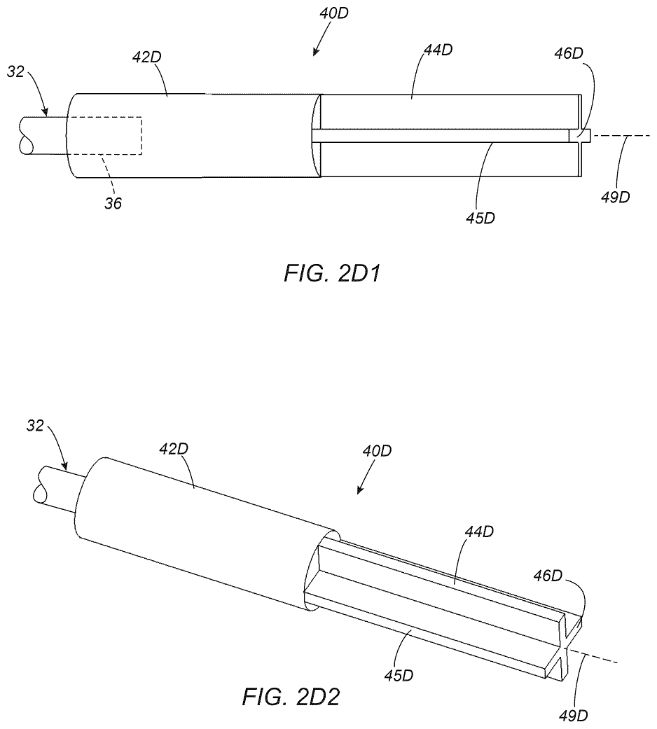

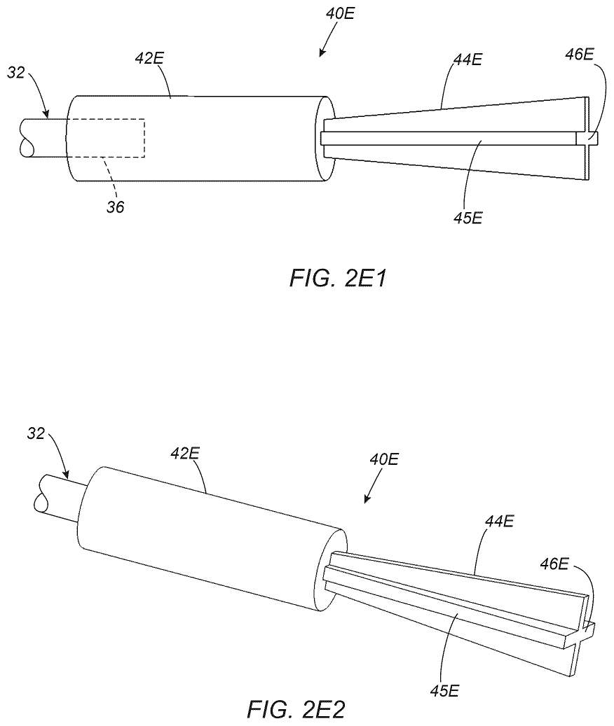

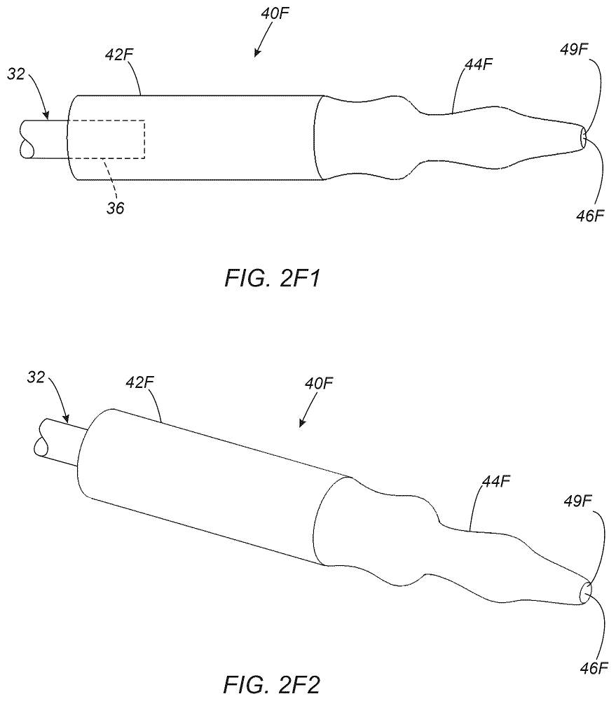

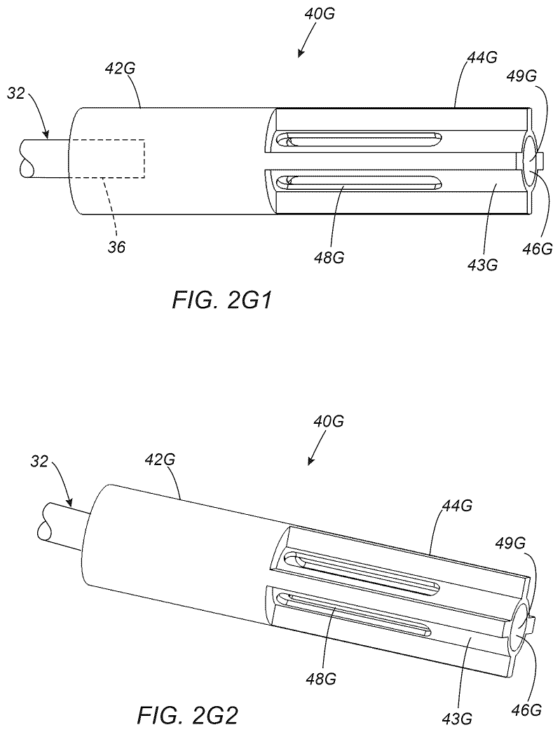

Generally, as shown in FIGS. 2A-2G, the spinner head 40 may include a hub 42 attached to the distal end 36 of the shaft 32 and one or more features 44 extending from the hub 42 that may interact with clot or other material aspirated into the lumen 26 of the catheter 20, e.g., to break up and/or otherwise debulk clot. The hub 42 may be substantially permanently attached the distal end 36 of the shaft 32, e.g., by one or more of over-molding, fusing, sonic welding, cooperating connectors, and the like. For example, the hub 42 may include a recess that may receive the distal end 36 of the shaft 32, and the hub 42 may be bonded, melted, press-fit, and/or otherwise permanently attached over the shaft 32. Alternatively, the spinner head may be omitted and the distal end 36 of the shaft 32 itself may be used to debulking and remove clot.

Optionally, the spinner device 30 may include one or more markers, e.g., radiopaque rings or deposited material, at desired locations, e.g., on the distal end 36 of the shaft 32 and/or on the spinner head 40, which may facilitate monitoring introduction and/or operation of the spinner device 30 during a procedure, e.g., using fluoroscopy, Xray, ultrasound, or other external imaging. In addition or alternatively, the shaft 32 may be constructed from radiopaque materials, which may facilitate monitoring the shaft 32 during introduction and/or manipulation during a procedure.

Turning to FIGS. 2A-2G, examples of spinner heads 40 are shown that may be used with the devices, systems and methods provided herein, e.g., on the distal end 36 of the shaft 32 (not shown, see FIG. 1). For example, in FIGS. 2A1 and 2A2, the spinner head 40A includes a hub 42A that may be attached to the distal end 36 of the shaft 32 and a plurality of blades or struts 44A extending axially from the hub 42A to a distal tip 46A of the spinner head 40A. As shown, the struts 44A extend substantially parallel to one another (and the longitudinal axis 28 of the apparatus 10 shown in FIG. 1).

In the example shown, the struts 44A may be coupled together at the distal tip 46A such that the struts 44A rotate together and do not move independently relative to one another. A slit or opening 48A may be provided between the struts 44A between the distal tip 46A and the hub 42A, which may generate localized suction when the spinner head 40A is rotated and/or that may capture or entangle material, e.g., fibrin material from a clot aspirated into the spinner head 40A.

Alternatively, as shown in FIGS. 2B1 and 2B2, the spinner head 40B may include a plurality of blades or struts 44B that extend axially from the hub 42B. Tips 46B of the struts 44B may be uncoupled from one another such that a cavity 48B extends from the tips 46B between the struts 44B. The struts 44B may be substantially stiff, e.g., having sufficient flexibility to allow the spinner head 40B to be advanced through the catheter 20 and/or otherwise through a patient's vasculature, while being substantially fixed relative to one another when the spinner head 40B rotates at high speeds. Alternatively, the struts 44B may be formed from flexible material to accommodate the tips 46B moving relative to one another while maintaining the struts 44B aligned with one another around the cavity 48B, i.e., such that the struts 44B do not become entangled one another.

Optionally, a transition 47B may be provided between the hub 42B and the struts 44B. For example, as shown, the transition 47B may taper inwardly from the hub 42B between proximal ends of the struts 44B. Such a transition may facilitate fragments of clot or other debris moving proximally past the spinner head 40, e.g., due to suction applied during use of the device, as described elsewhere herein.

In the examples shown in FIGS. 2A1-2B2, the spinner head 40A, 40B includes three struts 44A, 44B. Alternatively, the spinner head may include two, four, or more struts, if desired. For example, in FIGS. 2C1 and 2C2, a spinner head 40C is shown, which is generally similar to the spinner head 40B, except that four struts 44C are provided that extend from the hub 42C.

Turning to FIGS. 2D1 and 2D2, another example of a spinner head 40D is shown that includes a hub 42D attached to the distal end 36 of the shaft 32 and a plurality of struts 44D extending axially from the hub 42D. Unlike the previous spinner heads, the struts 44D are coupled together at inner edges such that the struts 44D extend radially outwardly from a central axis 49D of the spinner head 40D. Thus, the struts 44D do not have any inner space or cavity between them. In the example shown, the spinner head 40D includes four struts 44D extending outwardly from the central axis 49D such that the struts 44D are spaced equally around the axis 49D, e.g., about ninety degrees (90°) from one another, to define a cross-shaped cross-section. Alternatively, the spinner head may include fewer or more struts, e.g., two, three, five, six, or more, and/or the struts may be asymmetrically spaced from one another (not shown), if desired.

As best seen in FIG. 2D1, the struts 44D have a substantially uniform height (radially outwardly from the axis 49D to their outer edges 45D) along their lengths between the hub 42D and the distal tip 46D, e.g., such that the cross-shaped cross-section defines a single diameter along the length of the struts 44D. Alternatively, the struts may have a non-uniform height along their lengths.

For example, as shown in FIGS. 2E1 and 2E2, the spinner head 40E may include struts 44E that taper between the hub 42E and distal tip 46E. In the example shown, the struts 44E taper linearly inwardly from the distal tip 46E towards the hub 42E such that the outer edges 45E are substantially straight. Alternatively, if desired, the struts may have a non-linear taper between the distal tip and the hub and/or the struts may taper in the opposite direction, i.e., from the hub towards the distal tip.

Turning to FIGS. 2F1 and 2F2, another example of a spinner head 40F is shown that includes a hub 42F attached to the distal end 36 of the shaft 32, similar to other spinner heads herein. Unlike the other spinner heads, the spinner head 40F includes a spinner tip 44F having a variable diameter cylindrical cross-section, e.g., including a bulbous region adjacent the hub 42F and tapering to a distal tip 46F of the spinner head 40F. Optionally, as shown, the distal tip 46F may include an opening 49F extending into the spinner tip 44F, e.g., to slidably receive a guidewire (not shown). For example, the spinner head 40F and shaft 32 may include an axial lumen (not shown) that communicate with the opening 49F, e.g., such that a guidewire may be backloaded into the opening 49F and axial lumen to allow the spinner device 30 to be advanced into a target location over the guidewire. The guidewire may remain through the opening 49F during rotation of the spinner head 40F, e.g., to help stabilize the spinning motion to ensure spinner head 40F spins concentrically with the shaft 32.

Turning to FIGS. 2G1 and 2G2, yet another example of a spinner head 40G is shown that includes a plurality of struts 44G extending axially from a hub 42G. Unlike the previous spinner heads, the struts 44G extend outwardly from a cylindrical body 43G extending distally from the hub 42G. The cylindrical body 43G includes a cavity 49G extending from the distal tip 46G towards the hub 42G. Optionally, the cylindrical body 43G may include one or more slits or openings 48G, e.g., extending through the cylindrical body 43G into the cavity 49G to enhance localized suction. For example, an elongate slit 48G may be provided between each of the struts 44G. As shown, four struts 44G are shown that are spaced evenly around the cylindrical body 43G, e.g., offset about ninety degrees from one another around the circumference of the wall of the body 42B. Also as shown, the struts 44G may extend the length of the cylindrical body 43G or may only extend partially between the hub 42G and the distal tip 46G. It should be understood that while equal spacing is shown, alternatively, the struts 44G may be spaced unequally around the cylindrical body 43G. In addition, while four struts 44G are shown, any number of struts may be used, e.g., two, three, four, five, six, seven, etc. Similarly, it should be understood that the blades need not be of equal dimensions (e.g., length, width, height), but may have unequal dimensions, if desired.

In another alternative, the spinner heads herein may include non-axial struts extending at least partially between the hub and the distal tip. For example, a plurality of helical struts or blades may be provided (not shown), e.g., extending at least partially around the circumference of a cylindrical body and/or along the length between the hub and the distal tip of the spinner head. Optionally, in this alternative, one or more helical slits or other openings may be provided between one or more of the helical struts. It will be appreciated that one or more additional features may be provided on the outer surface of the spinner head, in addition to or instead of the struts and/or slits, if desired, e.g., to enhance localized suction generated by the spinner head. Additional examples of spinner heads that may be used with the devices, systems, and methods herein are disclosed in International Publication No. WO 2023/219965, the entire disclosure of which is expressly incorporated by reference herein.

With reference to FIGS. 2A1 and 2B1, the spinner head 40A (or any other examples shown in FIGS. 2A-2G or otherwise described herein) may be integrally formed as a single piece, e.g., from plastic, metal, composite material, and the like, e.g., manufactured using one or more of 3D-printing, by molding, micro-injection molding, casting, machining, and the like. Alternatively, the hub 42A and/or struts 44A may be formed separately from one or more substantially continuous processes, such as extrusion and the like and attached together to provide the final spinner head 40A, e.g., to allow manufacturing a single assembly that may be separated and that attached together into individual spinner heads.

The spinner head 40A may be substantially rigid or, alternatively, the material of the spinner head 40A may be flexible or semi-rigid, e.g., formed from relatively soft material, such as elastomeric material, e.g., silicone, or soft plastics, such that the distal tip 46A provides a substantially atraumatic tip that may minimize risk of damaging tissue contacted by the tip. For example, the spinner head 40A may be formed from relatively soft material, such as an elastomeric material with stiffness less than 40 MPa. Thus, the spinner head 40A may be able to recover from deformation and retain its shape, e.g., by bending less than about one hundred eighty degrees (180)° degrees and/or twisting less than about five hundred forty degrees (540°).

Returning to FIG. 1, the spinner head 40 is connected to the shaft 32, which is coupled, in turn, to the motor 60 that provides the torque to rotate the spinner head 40. For example, the proximal end 34 of the shaft 32 may include a connector (not shown) to couple the shaft 32 to the motor 60, e.g., via an external drive shaft, cable, and the like (also not shown). The motor 60 may be configured to rotate the spinner head 40 at desired speeds, e.g., at least one hundred rotations per minute (100 rpm), or at least 10,000 rpm, e.g., between about 1,000 and 200,000 rpm, between about 4,000 and 50,000 rpm, between about 10,000 and 40,000 rpm, between about 20,000 and 40,000 rpm, or between about 30,000 and 40,000 rpm, e.g., around 10,000 rpm or around 40,000 rpm.

In one example, the motor 60 may be configured to rotate the spinner head 40 at a single set speed. Alternatively, the speed of the motor 60 may be variable, e.g., manually using an actuator of the controller 62 coupled to the motor 60, which may be adjusted by the user to modify the rotation speed of the spinner tip 40. Alternatively, the controller 62 may be configured to initially operate the motor 60 at a relatively lower speed and then the speed may be automatically increased to rotate the spinner tip 40 at a desired active speed. For example, the initial speed may be used to mechanically engage the clot, and then the speed may be increased, e.g., to debulk and/or otherwise break up the clot, as described elsewhere herein.

Optionally, the controller 62 may include one or more actuators, e.g., switches and the like (not shown), to activate/deactivate the motor and/or to adjust the speed, if desired, e.g., to allow a user to turn the motor 60 off and on to rotate the spinner head 40. In addition or alternatively, the controller 62 may include an actuator (also not shown) to advance and/or retract the shaft 32 axially, e.g., relative to the catheter 20, e.g., using an advancer device (not shown). In another option, the controller 62 may include a robotic control system to control axial movement of the shaft 32 remotely, if desired.

In one example, the spinner device 30 and catheter 20 are assembled together, e.g., such that the catheter 20 and spinner device 30 are introduced together into a patient's body. In this way, the devices described herein are part of a pre-assembled system or kit. Alternatively, the spinner device 30 may be separate from the catheter 20, i.e., including the shaft 32 and spinner tip 40, e.g., such that the catheter 20 may be introduced initially into a patient's body, and, once the distal end 24 and outlet 25 are positioned adjacent a target clot, the spinner device 30 may be introduced into the catheter 20 and advanced to position the spinner head 40 adjacent the outlet 25. In this way, the devices described herein may be assembled just prior to, or during, use. In this example, the handle 50 of the catheter 20 may include a port 52a that allows the spinner device 30 to be inserted into and removed from the lumen 26 of the catheter 20, which may include one or more hemostatic seals, e.g., to prevent fluid from leaking from the port 52a while allowing the shaft 32 of the spinner device 30 to be advanced through the port 52a into the lumen 26. Alternatively, the spinner device 30 may be permanently integrated with the catheter 20, e.g., such that the spinner device 30 cannot be removed but may be advanced and/or retracted axially within the lumen 26, e.g., using the advancer device 150.

Optionally, the catheter 20 and/or shaft 32 may include one or more stops or other safety features (not shown) to limit axial movement of the shaft 32 and thus help prevent accidental shearing of non-clot tissue during use. For example, a stop may be provided within the handle 50 that prevents the spinner device 30 from being advanced to expose the spinner head 40 from the outlet 25 of the catheter 20. Optionally, another stop may be provided that allows the spinner head 40 to be retracted a desired distance proximally from the outlet 25, e.g., to allow residual fiber from a reduced or dissolved clot to be aspirated or otherwise directed into the outlet 25, as described further elsewhere herein. In another alternative, the spinner device 30 may be axially fixed relative to the catheter 20, e.g., such that the spinner head 40 is located within the lumen 26 with the distal end 46 of the spinner head 40 located immediately adjacent the outlet 25.

Alternatively, the spinner device 30 may be movable axially to advance the spinner head 40 from the distal end 24 of the catheter 20 beyond the outlet 25. For example, as described elsewhere herein, during use, the spinner head 40 may be advanced from the outlet 25 into or completely through a target clot, and then retracted to pull the clot through the outlet 25 into the lumen 26, e.g., while the spinner head 40 rotates to break up and/or entangle the clot. Optionally, in this alternative, the catheter 20 and/or spinner device 30 may include one or more stops to limit advancement of the spinner head 40 a predetermined distance out the outlet 25.

If the spinner device 30 is permanently integrated with the catheter 20, the proximal end 34 of the shaft 32 may extend from the port 52a on the handle 50 (whether the shaft 32 is movable axially or axially fixed). The proximal end 34 of the shaft 32 may include a connector configured to couple the shaft 32 to a driveshaft of the motor 60 (not shown), e.g., to allow the thrombectomy device 10 to be connected and disconnected from the motor 60. In this example, the thrombectomy device 10 may be a single-use, integral device that may be provided to a user for use during a procedure, after which the device 10 may be discarded. Alternatively, if the spinner device 30 is provided separately from the catheter 20, both may be single-use and/or disposable or one or both may be reusable, e.g., after cleaning and/or sterilization. In a further alternative, the spinner device 30 may be provided and/or introduced into a patient's body without the catheter 20, if desired.

With continued reference to FIGS. 1A and 1, the device 10 (or any of the devices, systems, and methods described herein) include a source of vacuum 64, e.g., a pump, syringe, suction line, and the like (not shown), that may be coupled to the proximal end 22 of the catheter 20, e.g., to port 52b on the handle 50 for aspirating material into the catheter lumen 26. For example, the port 52b may include a Luer fitting or other connector that allow tubing from the source of vacuum 64 to be removably connected to the port 52b.

Before or during advancing and/or activating the spinner head 40 within a blood vessel to debulk or otherwise remove a clot, the source of vacuum 64 may be activated to generate suction within the lumen 26. For example, as described further elsewhere herein, the vacuum source 64 may be activated after positioning the distal end of the catheter 20 adjacent a target clot but before activating the spinner head 40 to provide continuous aspiration into the lumen 26 to debulk and/or otherwise remove clot material adjacent the outlet 25 of the lumen 26 of the catheter 20.

In addition or alternatively, a source of fluid, e.g., a syringe of saline, contrast, and the like, may be connected to the port 52b (or to a separate dedicated port, not shown, if desired). Thus, during use of the catheter 20, the lumen 26 may be flushed and/or the fluid may be delivered through the outlet 25, if desired.

Optionally, the thrombectomy device 10 and/or spinner device 30 may be included in a system or kit including one or more additional devices for use during a thrombectomy procedure. For example, the system may include an occlusion device and/or a capture member (not shown) to prevent fragments of a clot being treated from migrating elsewhere within the patient's vasculature. For example, such devices may be introduced and deployed from the catheter 20, e.g., through the lumen 26 or a secondary lumen. Alternatively, such devices may be introduced and deployed independently of the spinner device, e.g., via a separate catheter, sheath, or other device (not shown) downstream from the target clot.

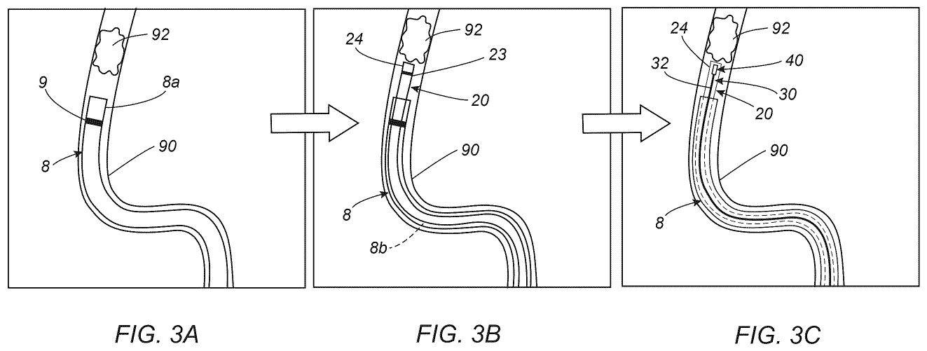

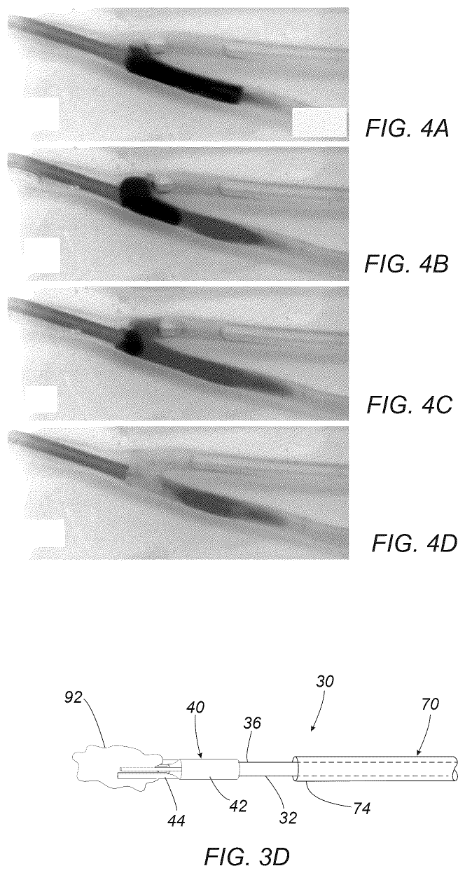

The devices and systems herein may be used during a thrombectomy procedure. For example, as shown in FIGS. 3A-3C, an exemplary method is shown for removing clot from within one or more blood vessels, e.g., a cerebral or cervical artery within a patient's body. Alternatively, the devices and systems herein may be used to remove debris or other material within other body lumens within a patient's body.

Initially, as shown in FIG. 3A, a guiding catheter 8 may be introduced into the patient's vasculature and advanced or otherwise manipulated to position the distal end 8a within a target vessel 90. The distal end 8a may be introduced into the patient's body from a percutaneous entry site into the patient's vasculature, e.g., over a guidewire or other rail and/or through an introducer sheath, guide sheath, or other tubular member (not shown).

Turning to FIG. 3B, the distal end 24 of the aspiration catheter 20 may then be introduced into the target vessel 90, e.g., through a lumen 8b of the guiding catheter 8. For example, the aspiration catheter 20 may be advanced through the guiding catheter 8 until the distal 24 is exposed and then manipulated to position the outlet 25 within a blood vessel 90 adjacent a target clot 92, e.g., as shown in FIG. 5A. Optionally, manipulation of the catheters 8, 20 may be monitored using external imaging, e.g., fluoroscopy, Xray, ultrasound, and the like. For example, the distal ends 8a, 24 of the guiding and aspiration catheters 8, 20 may include one or more radiopaque markers 9, 23 that may be monitored using fluoroscopy to facilitate positioning the distal ends 8a, 24 within the body lumen 90 and/or adjacent the clot 92. Optionally, contrast may be introduced into the blood vessel 90, e.g., via the guiding catheter lumen 8b, via the port 52b (shown in FIG. 1), a separate port on the proximal end 22 of the catheter 20, or a separate device (not shown), to facilitate locating the clot 92 and/or positioning the outlet 25 adjacent the clot 92. In addition or alternatively, the catheter 20 may be monitored using intravascular imaging systems and methods, such as intravascular ultrasound imaging, optical coherence tomography, and the like.

Turning to FIG. 3C (where the markers 9, 23 have been omitted for clarity), the spinner head 40 may be positioned within the lumen 26 of the catheter 20 adjacent the outlet 25. In one example, after introducing and positioning the catheter 20, the spinner head 40 and shaft 32 may be introduced into the lumen 26 from the proximal end 24 of the catheter 20, e.g., through port 52a, and advanced to position the spinner head 40 adjacent the outlet 25 of the catheter 20 and, consequently, adjacent the clot 92. Optionally, the spinner device 30 and/or catheter 20 may include one or more stops (not shown) that limit advancement of the spinner head 40, e.g., to prevent the spinner head 40 from being exposed through the outlet 25, as described elsewhere herein.

Optionally, manipulation of the spinner device 30 may be monitored using external imaging, e.g., fluoroscopy, Xray, ultrasound, and the like. For example, the spinner device 30 may include one or more radiopaque markers, e.g., on the distal end 36 of the shaft 32 and/or spinner head 40, and/or the shaft 32 may be formed from radiopaque material that may be monitored to facilitate positioning the spinner head 40 adjacent the outlet 25 and clot 92. Optionally, contrast may be introduced into the blood vessel 90, e.g., via the port 52b, a separate port on the proximal end 22 of the catheter 20, or a separate device (not shown), to facilitate locating the clot 92 and positioning the spinner head 40.

Alternatively, as described elsewhere herein, the spinner device 30 may be provided integral with the catheter 20 such that the spinner device 30 may be initially provided within the catheter 20, i.e., such that the spinner device 30 is introduced along with the catheter 20. The spinner device 30 may be movable axially relative to the catheter 20, e.g., by manipulating an actuator (not shown) on the handle or hub 50, to position the spinner head 40 as desired relative to the outlet 25. In a further alternative, the spinner device 30 may be axially fixed relative to the catheter 20, e.g., such that the spinner head 40 is axially fixed adjacent the outlet 25, i.e., such that the spinner head 40 is positioned adjacent the clot 92 at the same time as the distal end 24 of the catheter 20.

Once the spinner head 40 and outlet 25 are positioned as desired relative to the clot 92, the source of vacuum 64 may be activated to apply suction into the outlet 25 and lumen 26. For example, if the source 64 is a pump, the pump may be turned on or, if the source 64 is a suction line, the line may be opened, to begin applying continuous suction to the outlet 25, which may draw the clot against the outlet 25.

As shown in FIG. 3C, the spinner head 40 may then be rotated to debulk and/or otherwise remove the clot 92. For example, the motor 60 may be activated, e.g., using the controller 62, thereby causing the shaft 32 and spinner head 40 to rotate. As described elsewhere herein, the speed of the spinner head 40 may be fixed or adjustable, e.g., manually by the user and/or automatically by the controller 62.

Unlike other thrombectomy devices that are positioned adjacent or within a clot to catch or macerate the entire clot, the device 10 may direct the clot against and into the opening 25, whereupon the clot may engage the rotating spinner head 40, which may debulk or otherwise break up the clot such that the clot may be aspirated through the lumen 26 around the spinner device 30.

For example, with respect to the spinner head 40A shown in FIGS. 2A1 and 2A2, the clot may contact the spinning struts 44A as the clot enters the outlet 25 of the catheter 20, which may mechanically entangle and/or fragment the clot into pieces small enough to be aspirated through the lumen 26. Because the spinner head 40 remains within the lumen 26 of the catheter 20, as the clot breaks up, the continuous suction prevents fragments or other debris from escaping through the outlet 25.

In addition or alternatively, rotation of the spinner head 40 may rapidly separate red blood cells from the clot, leaving a fibrin fiber network that may be broken up or entangled with the struts 44A. If the entire clot is not broken up and aspirated, any residual fibrin or other material entangled by the spinner head 40 may be removed with the spinner device 30.

For example, if the spinner device 30 is removable from the catheter 20, while the motor 60 remains activated to rotate the spinner head 40, the proximal end 33 of the spinner device 30 may be pulled or otherwise manipulated to withdraw the spinner head 40 proximally through the lumen 26, thereby pulling the remaining material through the lumen 26 and out of the patient's body. Alternatively, the motor 60 may be deactivated at any time before or after removing the spinner device 30. The source of vacuum 64 may remain activated during the withdrawal to prevent material from escaping and flowing distally through the lumen 26 back out the outlet 25. Once the spinner device 30 is removed from the catheter 20, the source of vacuum 64 may be deactivated. FIG. 3D shows an example of a spinner head 40 including residual clot material captured by struts 44 of the spinner head, which have been removed from an aspiration catheter and a patient's body (not shown).

When the spinner head 40 is rotated while the suction is applied, the clot may be debulked and/or otherwise removed in as little as ten seconds or less. However, the spinner device 30 may remain activated for as long as desired to ensure the clot is removed from the target location 90. For example, the spinner device 30 may remain activated for several minutes while remaining adjacent the outlet 25 before withdrawing the spinner device 30.

Alternatively, the spinner device 30 may be movable axially to advance the spinner head 40 from the distal end 24 of the catheter 20 beyond the outlet 25. For example, after positioning the catheter 20 adjacent a target clot 92, the spinner head 40 may be advanced from the outlet 25 into or completely through the clot 92, and then retracted to pull the clot 92 through the outlet 25 into the lumen 26. The motor 60 may be activated to rotate the spinner head 40 after being positioned into or through the clot 92 to entangle the clot 92 to facilitate pulling the clot 92 into the lumen 24. Further rotation of the spinner head 40 may then break up the clot, which be aspirated through the lumen 24 by the suction. Optionally, in this alternative, the catheter 20 and/or spinner device 30 may include one or more stops to limit advancement of the spinner head 40 a predetermined distance out the outlet 25 to engage the clot 92.

In a further alternative, where the spinner device 30 is not removable from the catheter 20, but is movable axially, after activating the spinner device 30 to debulk and/or remove the clot 92 from the body lumen 90, the spinner device 30 may be manipulated proximally away from the outlet 25, e.g., to pull any residual clot material into the lumen 26 and away from the outlet 25 to prevent escape. For example, the spinner head 30 may be positioned within the lumen 26 immediate adjacent the outlet 25 to break up the clot 92 as it enters the lumen 26 or the spinner head 30 may be advanced into or through the clot and then withdrawn to pull the clot 92 into the lumen 26.

In this alternative, the entire device 10, i.e., the catheter 20 together with the spinner device 30, may be removed from the target location 90, e.g., into the guiding catheter 8 shown in FIGS. 3A-3C or independently back through the patient's vasculature and out of the patient's body.

FIGS. 4A-4D show an example of the changes in a blood clot (created by pig blood in this example) during spinning the spinner head of a thrombectomy device in ten seconds, with exemplary images after zero, seven, eight, and ten seconds. As can be clearly seen, the size of the clot significantly reduces, and the clot is removed into the catheter.

The clot and/or other debris aspirated through the lumen 26 of the catheter 20 may be collected outside the patient's body, e.g., within a container (not shown) communicating with the vacuum source 60.

While the invention is susceptible to various modifications, and alternative forms, specific examples thereof have been shown in the drawings and are herein described in detail. It should be understood, however, that the invention is not to be limited to the particular forms or methods disclosed, but to the contrary, the invention is to cover all modifications, equivalents and alternatives falling within the scope of the appended claims.

1. A thrombectomy device, comprising:
an elongated tubular member comprising a proximal end, a distal end sized for introduction into a body lumen of a patient, and a lumen extending between the proximal end and an outlet in the distal end;
a source of vacuum coupled to the proximal end and communicating with the lumen; and
a spinner device comprising:
a shaft comprising a first end and a second end sized for introduction into the lumen, thereby defining a longitudinal axis therebetween;
a spinner head on the second of the shaft positionable within the lumen adjacent the outlet within the lumen; and
a motor coupled to the first end of the shaft for rotating the shaft and spinner head around the axis to debulk or otherwise remove clot adjacent the distal end.

2. The device of claim 1, wherein the source of vacuum comprises a pump configured to be activated to apply vacuum to the lumen to aspirate debris from the clot through the lumen.

3. The device of claim 1, wherein the spinner device is configured to be introduced into the proximal end of the tubular member through the lumen to position the spinner head adjacent the outlet within the lumen.

4. The device of claim 3, further comprising one or more stops for limiting advancement of the spinner device to prevent the spinner head from being exposed through the outlet.

5. The device of claim 3, further comprising a handle or hub on the proximal end of the tubular member, and wherein the spinner device is configured to be introduced through a port on the handle or hub into the lumen.

6. The device of claim 1, wherein the spinner device is integral with the tubular member.

7. The device of claim 6, wherein the spinner device is movable axially relative to the catheter to move the spinner head between a distal position immediately adjacent the outlet and a proximal position spaced proximally from the outlet.

8. The device of claim 6, wherein the spinner head is axially fixed relative to the distal end of the tubular member.

9. The device of claim 1, further comprising a guiding catheter comprising a lumen sized to receive the distal end of the tubular member.

10. The device of claim 1, wherein the spinner head comprises a hub attached to the shaft and a plurality of struts extending distally from the hub.

11. The device of claim 10, wherein distal tips of the struts are fixed relative to one another.

12. The device of claim 11, wherein one or more slits are provided along the struts between the hub and the distal tip.

13. The device of claim 10, wherein distal tips of the struts are spaced apart from one another.

14. The device of claim 13, further comprising a cavity between the struts from the distal tips towards the hub.

15. The device of claim 1, wherein the spinner head comprises a variable cross-section cylindrical member extending from the second end of the shaft.

16. The device of claim 15, wherein the cylindrical member extends from a hub of the spinner head attached to the second end of the shaft.

17. The device of claim 16, wherein the cylindrical member comprises a bulbous region adjacent the hub and a tapered region that extends from the bulbous region to a distal tip of the spinner head.

18. (canceled)

19. The device of claim 1, wherein the spinner head comprises an annular body extending distally from the hub and wherein the struts extend outwardly from the annular body, the annular body comprising an opening communicating with a cavity within the annular body at the distal tip of the spinner head.

20-27. (canceled)

28. A thrombectomy device, comprising:
an elongated tubular member comprising a proximal end, a distal end sized for introduction into a body lumen of a patient, and a lumen extending between the proximal end and an outlet in the distal end;
a source of vacuum coupled to the proximal end and communicating with the lumen; and
a spinner device comprising:
a shaft comprising a first end and a second end sized for introduction into the lumen, thereby defining a longitudinal axis therebetween;
a spinner head on the second of the shaft positionable within the lumen and movable distally to extend the spinner head through the outlet into or through a target clot and proximally to pull the clot into the lumen; and
a motor coupled to the first end of the shaft for rotating the shaft and spinner head around the axis to debulk or otherwise remove clot adjacent the distal end.

29-49. (canceled)

50. A method for performing thrombectomy, comprising:
introducing a distal end of a tubular member into a body lumen adjacent a target blood clot;
positioning a spinner head within a lumen of the tubular member adjacent an outlet in the distal end;
activating a source of vacuum communicating with the lumen to aspirate material into the outlet; and
rotating the spinner head to debulk and/or otherwise remove the clot into the outlet and aspirate the clot through the lumen.

51-55. (canceled)
